(19) United States
(12) Patent Application Publication
(10) Pub. No.: US 2003/0162945 A1
(43) Pub. Date: Aug. 28, 2003

(54) G- protein coupled receptor org10

(76) Inventors: Morag Grassie, Cambuslang (GB); Emma Jane Grant, Flat (GB); Johannes Petrus Gerardus Klomp, Moergestel (NL)

Correspondence Address: William M Blackstone, Akzo Nobel, Patent Department, Intervet, 405 State Street, Millsboro, DE, 19966, US

(21) Appl. No.: 10/312,094

(22) PCT Filed: Jun. 29, 2001
(86) PCT No.: PCT/EP01/07018

(51) Int. Cl. C12Q 1/68; C07H 21/04; C12P 21/02; C07K 14/705; C12N 5/06

(52) U.S. Cl. 530/350; 536/23.5; 435/69.1; 435/6; 435/320.1; 435/325

(57) ABSTRACT

The present invention provides a full length cDNA sequence that codes for a G-protein coupled receptor, as well as the complete gene and the encoded protein. The present invention provides a recombinant cell line expressing these receptors at appropriate levels such that novel compounds active at these receptors may be identified for therapeutic use. The receptor sequence described in this invention is a member of a novel GPCR receptor sub-family which has no known endogenous ligand. This cDNA can be used to identify novel compounds active at the receptor for therapeutic use especially in the field of CNS disorders, neurodegeneration or analgesia.

The present invention provides a full length cDNA sequence that codes for a G-protein coupled receptor, as well as the complete gene and the encoded protein. The present invention provides a recombinant cell line expressing these receptors at appropriate levels such that novel compounds active at these receptors may be identified for therapeutic use. The receptor sequence described in this invention is a member of a novel GPCR receptor sub-family which has no known endogenous ligand. This cDNA can be used to identify novel compounds active at the receptor for therapeutic use especially in the field of CNS disorders, neurodegeneration or analgesia.

The G-protein coupled receptor (GPCR) superfamily is one of the largest protein families identified to date. This family comprises over 800 cloned members from a wide range of species, and includes at least 300 human members. GPCRs have a proven history as excellent therapeutic targets with between 40-50% of drug targets to date being GPCRs (Murphy et al., 1998). GPCRs are responsive to a wide variety of stimuli and chemical transmitters, including light, biogenic amines, amino acids, peptides, lipids, nucleosides, and large polypeptides. This results in the regulation of multiple processes including neurotransmission, cellular metabolism, secretion, cellular differentiation and growth as well as inflammatory and immune responses. Many of these GPCRs are expressed in the brain and may be exploited as therapeutic targets for the treatment of CNS disorders. More significantly, many GPCRs with no known endogenous ligands are still being identified in the public and proprietary databases. These orphan GPCRs represent potential novel therapeutic targets for a range of therapeutic intervention and the treatment of a variety of disorders

Reverse pharmacology or functional genomics is currently being adopted within the drug discovery process. This is gene-based biology which aims to pharmacologically validate novel genes by either identifying surrogate ligands or their endogenous ligand. This invention describes the use of this novel GPCR for the identification of novel therapeutic compounds.

There is evidence to suggest that in addition to novel orphan GPCRs, there also exist novel GPCR gene sub-families that bind previously unidentified ligands. Because many orphan GPCRs await to be assigned a natural ligand, many of these receptors may bind novel ligands which have not thus far been identified. (Civelli et al., 1999).

Orphan GPCRs are predicted to bind ligands, as it is postulated that inactive receptors should have been evolutionary discarded. Orphan receptors may therefore be used as baits to isolate their natural ligands or surrogate ligands. The use of this strategy in identifying novel ligands is exemplified in the identification of orphanin/nociceptin, orexins/hypocretins and prolactin-releasing peptide (Reinscheid et al., 1995, Sakurai et al., 1998 and Hinuma et al., 1998).

Many known G protein coupled receptors (GPCRs) are well established drug targets with a significant number of currently available drugs targeting such GPCRs (Wilson et al., 1998). Following activation of a GPCR by ligand binding to the receptor, the signal is amplified through a range of signal transduction cascades and consequently, regulation of this signal transduction pathway via a ligand binding to a GPCR offers the facility to modulate a tightly controlled biological pathway.

GPCRs mediate a wide range of biologically relevant processes and are responsive to a wide variety of stimuli and chemical/neurotransmitters, including light, biogenic amines, amino acids, peptides, lipids, nucleosides, and large polypeptides. How the cloning of a particular receptor has lead to the development of a therapeutic compound is particularly exemplified in the case of the serotonin/adrenergic receptors. Additionally a number of diseases are reported to be associated with mutations in known GPCRs (Wilson et al., 1998). The signalling pathways that mediate the actions of GPCRs have also been implicated in many biological processes significant to the pharmaceutical industry. Such signalling pathways involve G proteins, second messengers such as cAMP or calcium (Lefkowitz, 1991), effector proteins such as phospholipase C, adenylyl cyclase, RGS proteins, protein kinase A and protein kinase C (Simon et al., 1991).

For example a GPCR can be activated by a ligand, binding to the receptor resulting in the activation of a G protein which conveys the message onto the next component of the signal transduction pathway. Such a component could be adenylyl cyclase. In order for activation of this enzyme, the relevant G protein, of which there is a family, must exchange GTP for GDP, which is bound when the G protein is in an inactive state. The exchange of GDP for GTP can occur following the binding of ligand to the GPCR, however, some basal exchange of GDP for GTP can also occur depending on the receptor under investigation.

The conversion of GTP bound at the G protein to GDP occurs by hydrolysis and is catalysed by the G protein itself. Following this hydrolysis the G protein is returned to its inactive state. Consequently, the G protein mediates the transfer of the signal from the activated receptor to the intracellular signalling pathway, but also introduces an additional level of control, by controlling the length of time which the receptor can activate the intracellular signalling pathway through the GTP bound G protein.

In general the topology of these receptors is such that they contain 7 transmembrane domains consisting of approximately 20-30 amino acids. Consequently, these receptors are frequently known as 7TM receptors. These 7TM domains can be defined by consensus amino acid sequences and by structural prediction algorithms. Within the putative transmembrane domains, hydrophobic helixes are formed which are connected via extracellular and intracellular loops. The N-terminal end of the polypeptide is on the exterior face of the membrane with the C-terminal on the interior face of the membrane.

A number of additional features are frequently observed in GPCRs. These include glycosylation of the N-terminal tail. A conserved cysteine in each of the first two extracellular loops, which are modified such that disulphide bonds are formed, which is believed to result in a stabilised functional tertiary structure. Other modifications which occur on GPCRs include lipidation (eg palmityolation and famesylation) and phosphorylation often in the C terminal tail. Most GPCRs also have sites for phosphorylation in the third intracellular loop, a region, which is believed to contribute to G protein interactions and signal transduction. Phosphorylation of the third intracellular loop by specific receptor kinases such as CAMP dependent protein kinase (cAPK) or a class of GPCR kinases (GRKs) in several GPCRs such as β-adrenoreceptor also mediates in the desensitization of such a receptor. Consequently, specific mutations in particular regions of the GPCR can have functional significance. GRKs are known to phosphorylate GPCRs on multiple sites with threonine and serine residues as targets. The phosphorylation not only inactives the receptor but also allows the receptor with an additional inhibitory protein known as β-arrestin. This interaction can also be used as an indication that the GPCR in question has been activated.

Although as yet only limited three-dimensional crystal structure data is available for GPCRs some details of the ligand binding site present on GPCRs have been reported. For some receptors the ligand binding sites are believed to comprise hydrophilic pockets formed by some of the transmembrane domains. Within the transmembrane domain, the amino acid within the α-helical structure align themselves such that the hydrophilic surface of the amino acid is facing inwards towards the centre of the ligand binding pocket. This results in a postulate polar ligand binding site. The third transmembrane domain of has been reported to be involved in ligand binding in several GPCRs. In particular the aspartate of TM3, serines of TM5, asparagine of TM6 and phenylalanine or tyrosines of TM6 and/or TM7 have been implicated in ligand binding.

In addition to activating intracellular signalling pathways, GPCRs can also couple via G proteins to additional gene families such as ion channels, transporter and enzymes. Many GPCRs are present in mammalian systems exhibiting a range of distribution patterns from very specific to very widespread. For this reason following the identification of a putative novel GPCR by bioinformatics, assigning a therapeutic application to the novel GPCR is not obvious due to this diverse function and distribution of previously reported GPCRs.

There is clearly a need to identify and characterise novel GPCRs that can function to alter disease status either correction, prevention or amelioration. Such disease are diverse and include but are not exclusive to depression, schizophrenia, anxiety, neurological disorders, obesity, insomnia, addiction, neurodegeneration, hypotension, hypertension, acute heart failure, atherothrombosis, atherosclerosis and osteoporosis.

The present invention provides a brain expressed gene/protein which we termed ORG10 and which was found to be preferentially expressed in the brain. Analysis of the protein provides evidence that it is a GPCR. The gene may therefore be used in conventional expression systems in order to select compounds that specifically react with ORG10. These compounds may then be used to treat CNS disorders.

The present invention relates to ORG10, in particular ORG10 polypeptides, ORG10 polynucleotides, recombinant materials and methods of their production. Additionally the invention relates to methods which for such polypeptides and polynucleotides can be used to identify compounds such as agonists, partial agonists, inverse agonists or antagonists active at the invention for treatment of disease, such as psychiatric diseases and neurodegeneration, in particular bipolar and unipolar disorders, schizophrenia and anxiety. Use of ligands active at the said invention may be used to correct diseases associated with an imbalance of ORG10 and associated pathways. In particular, this invention relates to a diagnostic assay for identifying modifications in ORG10 gene or expression associated with CNS diseases and especially preferred for bipolar depression, schizophrenia and affective disorders.

The complete cDNA sequence of ORG10 shown in SEQ ID NO: 4 was translated which resulted in the amino acid sequence of SEQ ID NO: 3 which was then compared with known protein sequences. ORG10 was found to be related to a previously reported GPCR accession number g992582.

The genomic sequence of ORG10 is provided in SEQ ID NO: 1.

A fragment of ORG10 cDNA encompassing approximately 800 bp of the open reading frame was used to probe a human Multiple Tissue Expression (MTE) array (Clontech) containing human mRNA samples from 76 different tissues. The experimental procedure employed is described below. Analysis of the MTE array revealed that ORG10 is highly expressed in the corpus callosum, caudate nucleus, putamen and spinal cord, with lower levels of expression exhibited in other brain regions such as the amygdala and hippocampus. As ORG10 is highly expressed in the basal ganglia and corpus callosum this pattern of distribution makes it likely that this orphan receptor is linked to disorders of the basal ganglia, e.g. movement disorders, neurodegeneration or other impairment of motor function. In addition, the expression of ORG10 in the corpus callosum, which has a large population of Schwann cells, may also implicate a role for ORG10 in neurodegenerative disorders. Therefore expressing this receptor in a recombinant cell line allows screening to identify chemical entities which bind to this receptor. These compounds may then be used to treat Parkinson's disease, Huntington's Chorea, Gilles de la Tourette's syndrome, dystonia, obsessive compulsive disorder and schizophrenia as well as other CNS and neurodegenerative diseases. Peripherally, ORG10 appears to be expressed at much lower levels in a variety of tissues including the heart and skeletal muscle.

As GPCRs have key sequence motifs, aligning the 7 transmembrane domains of orphan and non-orphan GPCRs can create a phylogenetic tree BLAST analysis revealed that the full length ORG10 possesses identity with the human serotonin 5HT6 receptor indicating potential therapeutic uses of ORG10 ligands in the fields of psychiatry, obesity and diabetes, analgesia and anaesthesia.

The 5HT6 receptor has been proposed to be involved in antipsychotic drug action, with various psychotics and antidepressants having a high affinity for this receptor (Meltzer, 1999). Additionally 5HT6 also appears to be exclusively expressed in brain. Polymorphism in this gene have also been proposed to contribute to the genetic background of schizophrenia (Shinkai et al., 1999) and may contribute to the responsiveness of patients to clozapine, an antipsychotic, in particular those patients with anxious or depressed symptoms (Yu et al., 1999). Due to the homology that ORG10 displays with this receptor, identification of substances acting at ORG10 are anticipated to have therapeutic benefits in the areas of either psychiatry or neurology.

The genomic polynucleotides of the present invention may be obtained using standard cloning and screening techniques from a human genomic DNA library, however a full length cDNA product lacking the non-coding region of the invention as described in SEQ ID NO: 1 can only be obtained from a cDNA library such as a brain cDNA library. Polynucleotides detailed in this invention could also be generated from genomic DNA or synthesized using well known and commercially available techniques.

The sequences of the present invention can be used to derive primers and probes for use in DNA amplification reactions in order to perform diagnostic procedures or to identify further, neighbouring genes that also may contribute to the development of CNS disorders. The sequences of the present invention may also be used to design oligonucleotide probes for more detailed tissue distribution information by in situ hybridisation analysis. In addition, the sequences of the present invention may also be used to design oligonucleotide probes for use in antisense technology

It is known in the art that genes may vary within and among species with respect to their nucleotide sequence. The ORG10 genes from other species may be readily identified using the above probes and primers. Therefore, the invention also comprises functional equivalents, which are characterised in that they are capable of hybridising to at least part of the ORG10 sequence shown in SEQ ID NO: 1, preferably under high stringency conditions.

Two nucleic acid fragments are considered to have hybridisable sequences if they are capable to hybridising to one another under typical hybridisation and wash conditions, as described, for example in Maniatis, et al., pages 320-328, and 382-389, or using reduced stringency wash conditions that allow at most about 25-30% basepair mismatches, for example: 2×SSC, 0.1% SDS, room temperature twice, 30 minutes each, then 2×SSC, 0.1% SDS 37° C. once, 30 minutes; then 2×SSC, room temperature twice ten minutes each. Preferably, homologous nucleic acid strands contain 15-25% basepair mismatches, even more preferably 5-15% basepair mismatches. These degrees of homology can be selected by using wash conditions of appropriate stringency for identification of clones from gene libraries or other sources of genetic material, as is well known in the art.

Furthermore, to accommodate codon variability, the invention also includes sequences coding for the same amino acid sequences as the sequences disclosed herein. Also portions of the coding sequences coding for individual domains of the expressed protein are part of the invention as well as allelic and species variations thereof. Sometimes, a gene expresses different isoforms in a certain tissue which includes splicing variants, that may result in an altered 5′ or 3′ mRNA or in the inclusion of an additional exon sequence. Alternatively, the messenger might have an exon less as compared to its counterpart. These sequences as well as the proteins encoded by these sequences all are expected to perform the same or similar functions and form also part of the invention.

The sequence information as provided herein should not be so narrowly construed as to require inclusion of erroneously identified bases. The specific sequence disclosed herein can be readily used to isolate further genes which in turn can easily be subjected to further sequence analyses thereby identifying sequencing errors. Thus, in one aspect, the present invention provides for isolated polynucleotides encoding a novel gene, disrupted in schizophrenia.

The DNA according to the invention may be obtained from cDNA. Alternatively, the coding sequence might be genomic DNA, or prepared using DNA synthesis techniques. The polynucleotide may also be in the form of RNA. The polynucleotide may be in single stranded or double stranded form. The single strand might be the coding strand or the non-coding (anti-sense) strand.

The present invention further relates to polynucleotides which have at least 80%, preferably 90% and more preferably 95% and even more preferably at least 98% identity with SEQ ID NO:1. Such polynucleotides encode polypeptides which retain the same biological function or activity as the natural, mature protein.

“Identity” is the term given in the art, to the degree of similarity or relationship between 2 sequences determined by direct comparison along the length of those 2 sequences. There are numerous algorithms and computer packages available for the automated calculation of percentage identity between pairs of DNA sequences. These algorithms and computer applications may also compare a single sequence with all the members of a sequence database to determine which sequence in that database has the highest identity.

One such typical computer application is DNAMAN (Lynnon Biosoft, version 3.2). Using this program two sequences can be aligned using the optimal alignment algorithm of Smith and Waterman (1981). After alignment of the two sequences the percentage identity can be calculated by dividing the number of identical nucleotides between the two sequences by the length of the aligned sequences.

For comparison of polypeptide sequences the algorithm of Smith and Waterman above, may also be used, with the preferred parameter set known as the BLOSUM62 matrix. as given by Henikoff and Henikoff (1992). The well-known BLAST (Altschul et al., 1990) and FASTA (Pearson, 1990) suites of computer programs may also be used.

The DNA according to the invention will be very useful for in vivo or in vitro expression of the novel gene according to the invention in sufficient quantities and in substantially pure form.

In another aspect of the invention, there are provided polypeptides comprising the amino acid sequence encoded by the above described DNA molecules.

Preferably, the polypeptides according to the invention comprise at least part of the amino acid sequences as shown in SEQ ID NO: 3.

Also functional equivalents, that is polypeptides homologous to SEQ ID NO: 3 or parts thereof having variations of the sequence while still maintaining functional characteristics, are included in the invention.

The variations that can occur in a sequence may be demonstrated by (an) amino acid difference(s) in the overall sequence or by deletions, substitutions, insertions, inversions or additions of (an) amino acid(s) in said sequence. Amino acid substitutions that are expected not to essentially alter biological and immunological activities, have been described. Amino acid replacements between related amino acids or replacements which have occurred frequently in evolution are, inter alia Ser/Ala, Ser/Gly, Asp/Gly, Asp/Asn, Ile/Val (see Dayhoff). Based on this information Lipman and Pearson developed a method for rapid and sensitive protein comparison and determining the functional similarity between homologous polypeptides. It will be clear that also polynucleotides coding for such variants are part of the invention.

The polypeptides according to the present invention include the polypeptides comprising SEQ ID NO:3 but also their isoforms, i.e. polypeptides with a similarity of 70%, preferably 90%, more preferably 95%. Also portions of such polypeptides still capable of conferring biological effects are included. Especially portions which still bind to ligands form part of the invention. Such portions may be functional per se, e.g. in solubilized form or they might be linked to other polypeptides, either by known biotechnological ways or by chemical synthesis, to obtain chimeric proteins. Such proteins might be useful as therapeutic agent in that they may substitute the gene product in individuals with aberrant expression of the ORG10 gene.

The sequence of the gene may also be used in the preparation of vector molecules for the expression of the encoded protein in suitable host cells. A wide variety of host cell and cloning vehicle combinations may be usefully employed in cloning the nucleic acid sequence coding for the ORG10 gene of the invention or parts thereof. For example, useful cloning vehicles may include chromosomal, non-chromosomal and synthetic DNA sequences such as various known bacterial plasmids and wider host range plasmids and vectors derived from combinations of plasmids and phage or virus DNA.

Vehicles for use in expression of the genes or a ligand-binding domain thereof of the present invention will further comprise control sequences operably linked to the nucleic acid sequence coding for a ligand-binding domain. Such control sequences generally comprise a promoter sequence and sequences which regulate and/or enhance expression levels. Of course control and other sequences can vary depending on the host cell selected.

Suitable expression vectors are for example bacterial or yeast plasmids, wide host range plasmids and vectors derived from combinations of plasmid and phage or virus DNA. Vectors derived from chromosomal DNA are also included. Furthermore an origin of replication and/or a dominant selection marker can be present in the vector according to the invention. The vectors according to the invention are suitable for transforming a host cell.

Recombinant expression vectors comprising the DNA of the invention as well as cells transformed with said DNA or said expression vector also form part of the present invention.

Suitable host cells according to the invention are bacterial host cells, yeast and other fungi, plant or animal host such as Chinese Hamster Ovary cells or monkey cells. Thus, a host cell which comprises the DNA or expression vector according to the invention is also within the scope of the invention. The engineered host cells can be cultured in conventional nutrient media which can be modified e.g. for appropriate selection, amplification or induction of transcription. The culture conditions such as temperature, pH, nutrients etc. are well known to those ordinary skilled in the art.

The techniques for the preparation of the DNA or the vector according to the invention as well as the transformation or transfection of a host cell with said DNA or vector are standard and well known in the art, see for instance Sambrook et al. (1989).

The proteins according to the invention can be recovered and purified from recombinant cell cultures by common biochemical purification methods including ammonium sulfate precipitation, extraction, chromatography such as hydrophobic interaction chromatography, cation or anion exchange chromatography or affinity chromatography and high performance liquid chromatography. If necessary, also protein refolding steps can be included.

ORG10 gene products according to the present invention can be used for the in vivo or in vitro identification of novel ligands or analogues thereof. For this purpose binding studies can be performed with cells transformed with DNA according to the invention or an expression vector comprising DNA according to the invention, said cells expressing the ORG10 gene products according to the invention.

Alternatively also the ORG10 gene products according to the invention as well as ligand-binding domains thereof can be used in an assay for the identification of functional ligands, endogenous ligands or analogues for the ORG10 gene products.

Methods to determine binding to expressed gene products as well as in vitro and in vivo assays to determine biological activity of gene products are well known. In general, expressed gene product is contacted with the compound to be tested and binding, stimulation or inhibition of a functional response is measured.

Thus, the present invention provides for a method for identifying ligands for ORG10 gene products, said method comprising the steps of:

a) introducing into a suitable host cell a polynucleotide according to the invention,

b) culturing cells under conditions to allow expression of the DNA sequence

c) optionally isolating the expression product

d) bringing the expression product (or the host cell from step b)) into contact with potential ligands which will possibly bind to the protein encoded by said DNA from step a);

e) establishing whether a ligand has bound to the expressed protein.

f) Optionally isolating and identifying the ligand

As a preferred way of detecting the binding of the ligand to the expressed protein, also signal transduction capacity may be measured.

The present invention thus provides for a quick and economic method to screen for therapeutic agents for the prevention and/or treatment of diseases related to CNS disorders. The method is especially suited to be used for the high throughput screening of numerous potential ligands.

Compounds which activate or inhibit the function of ORG10 gene products may be employed in therapeutic treatments to activate or inhibit the polypeptides of the present invention.

Also within the scope of the invention are antibodies, especially monoclonal antibodies raised against the polypeptide molecule according to the invention. Such antibodies can be used therapeutically to inhibit ORG10 gene product function and diagnostically to detect ORG10 gene products.

The invention furthermore relates to the use of the ORG10 gene products as part of a diagnostic assay for detecting psychiatric abnormalities or susceptibility to psychiatric disorders related to mutations in the nucleic acid sequences encoding the ORG10 gene. Such mutations may e.g. be detected by using PCR (Saiki et al., 1986). Also the relative levels of RNA can be determined using e.g. hybridization or quantitative PCR technology. The presence and the levels of the ORG10 gene products themselves can be assayed by immunological technologies such as radioimmuno assays, Western blots and ELISA using specific antibodies raised against the gene products. Such techniques for measuring RNA and protein levels are well known to the skilled artisan.

The determination of expression levels of the ORG10 gene products in individual patients may lead to fine tuning of treatment protocols.

Also, transgenic animals may be prepared in which the expression of the ORG10 gene is altered or abolished.

### EXAMPLES

PCR Amplification of ORG10 cDNA

Full and partial cDNA encoding ORG10 were amplified by PCR using proof reading Expand polymerase (Roche) and AmplitaqGold Taq DNA polymerase (Perkin Elmer) respectively, and oligonucleotide primers based upon the sequence of ORG10. The template DNA used for the PCR reactions was Marathon-ready human whole brain cDNA (Clontech). The cycling conditions used in all reactions were identical, differing only in annealing temperature, and were as follows:

Following an initial denaturation step at 94° C. for 5 minutes, the reaction was allowed to cycle 33 times through a sequence of temperatures: 1) Denaturation of template 94° C. for 1 minute, 2) primer annealing at 55° C., for 1 minute 30 seconds, 3) DNA polymerisation at 72° C. for 2 minutes. A final elongation step at 72° C. for 10 minutes was also performed to ensure generation of full-length products.

Cloning of Full Length ORG10

The full length ORG10 cDNA generated in the PCR reaction described above was ligated into the prokaryotic expression vector pCR 2.1 (Invitrogen). Following chemical transformation and mini-prep DNA isolation, restriction digestion was performed using the restriction endonuclease EcoR I to excise the ORG10 cDNA from the vector. This restriction enzyme was used as the vector pCR 2.1 contains two EcoR I sites on either side of the multiple cloning site, which enable the subcloned insert to be excised effectively

Sequence Confirmation of Full Length ORG10

Following confirmation by restriction digestion, full-length ORG10 clones were selected for sequence analysis. The selected ORG10 clones were completely sequenced in both directions using the Big Dye terminator cycle sequencing kit and ABI310 Genetic Analyser (PE Biosystems) and following the manufacturers instructions. The primers used to sequence the selected clones were M13 forward and reverse and internal forward and reverse ORG10 specific primers.

Northern Analysis of ORG10

The relative abundance of ORG10 in a broad range of human tissues was analysed using a Multiple Tissue Expression (MTE) array purchased from Clontech. The ORG10 cDNA was amplified by PCR using the ORG10 5′ primer and internal reverse primer which produced a 800 bp probe corresponding to the 5′ most end of the cDNA. The PCR product was purified on QIAquick columns (Qiagen) and the DNA concentration was estimated by agarose gel electrophoresis. The cDNA (100 ng) was radiolabelled using the High Prime DNA labelling method (Boehringer Mannheim), and the probe was subsequently purified away from unincorporated nucleotides using ProbeQuant G-50 micro columns (Amersham Pharmacia Biotech). A prehybridisation mixture was prepared containing 10 ml of prewarmed ExpressHyb solution (Clontech) and 1 mg of sheared salmon sperm DNA (Sigma). The salmon sperm DNA was heat-denatured at 100° C. for 10 min and chilled quickly on ice prior to its addition to the ExpressHyb solution. The MTE array was placed in a Hybaid glass tube (25 cm) and the ExpressHyb mixture was added immediately. Prehybridisation was performed for 2 hours at 65° C. with continuous rotation. The purified labelled ORG10 cDNA probe was heat-denatured at 100° C. for 5 min and then combined with 1 ml of pre-hybridisation mixture, before adding this back to the remaining hybridisation solution in the glass tube. Hybridisation was performed overnight at 65° C. with continuous rotation. The MTE array was subjected to a series of washing steps as follows; four 20 min washes at 65° C. in 2×SSC plus 1% SDS, two 20 min washes at 55° C. in 0.1×SSC plus 0.5% SDS, and a final 30 min wash at 65° C. in 0.1×SSC plus 0.5% SDS. All washing steps were performed with continuous agitation in a large plastic containers with a lid. The MTE was wrapped in Saran wrap and exposed to X-ray film with an intensifying screen at −70° C. overnight.

Mammalian Cell Expression

The receptors of the present invention are expressed in e.g human embryonic kidney 293 (HEK293) cells or adherent CHO cells. To maximise receptor expression, typically all 5′ and 3′ untranslated regions (UTRs) are removed from the receptor cDNA prior to insertion into an appropriate eukaryotic expression vector e.g pcDNA3, including optional addition of Kozak 5′ consensus sequence. The cells are transfected with individual receptor cDNAs by lipofectin and selected in the presence of selected antibiotic (e.g hygromycin). After 3 weeks of selection, individual clones are picked and expanded for further analysis. HEK293 or CHO cells transfected with the vector alone serve as negative controls. To isolate cell lines stably expressing the individual receptors, about 24 clones are typically selected and analysed by Northern blot analysis. Receptor mRNAs are generally detectable in about 50% of the antibiotic resistant clones analysed.

Microphvsiomietric Assays

Activation of a wide variety of second messenger systems results in extrusion of small amounts of acid from a cell. The acid formed is largely as a result of the increased metabolic activity respired to fuel the intracellular signalling process. The pH changes in the media surrounding the cell are very small but are detectable by the CYTOSENSOR microphysiometer (Molecular Devices Ltd, Menlo Park, Calif.). The CYTOSENSOR is thus capable of detecting the activation of a receptor which is coupled to an energy utilising intracellular signalling pathway such as the G-protein coupled receptor of the present invention.

Extract/Cell Supematant Screening

A large number of mammalian receptors exist for which there remains, as yet, no cognate activating ligand (agonist). Thus, active ligands for these receptors may not be included within the ligand banks as identified to date. Accordingly, the 7TM receptor of the invention is also functionally screened (using calcium, cAMP, microphysiometer, oocyte electrophysiology, etc., functional screens) against tissue extracts to identify natural ligands. Extracts that produce positive functional responses can be sequentially subfractionated until an activating ligand is isolated and identified.

Calcium and cAMP Functional Assays

7TM receptors which are expressed in e.g HEK293 or CHO cells have been shown to be coupled functionally to activation of PLC and calcium mobilisation and/or cAMP stimulation or inhibition. Basal calcium levels in the HEK293 or CHO cells in receptor-transfected or vector control cells were observed to be in the normal, 100 nM to 200 nM range. HEK293 or CHO cells expressing recombinant receptors are loaded with fura 2 and in a single day >150 selected ligands or tissue/cell extracts are evaluated for agonist induced calcium mobilisation. Similarly, HEK293 or CHO cells expressing recombinant receptors are evaluated for the stimulation or inhibition of cAMP production using cAMP quantitation assays. Agonists presenting a calcium transient or cAMP fluctuation are tested in vector control cells to determine if the response is unique to the transfected cells expressing receptor. Additional detection methods for functional receptor activation by cAMP stimulation/inhibition and/or calcium mobilisation may include readouts from reporter gene constructs e.g luciferase luminescence and secreted alkaline phosphatase (SEAP).

Tissue Distribution Analysis of ORG10 by PCR

In order to further analyze the expression of the G-protein coupled receptor comprising SEQ ID NO: 1 in material from a variety of human tissues, PCR was performed using primers designed against the predicted ORG10 cDNA sequence (ORG10 forward primer 5′-ATG GCC MC TCC ACA GGG CTG 3′ (1-21, where 1=ATG translation initiation site) and ORG10 reverse primer 5′-GGA GAG AGA ACT CTC AGG TGG CCC 3′ (1104-1080). Each PCR contained 1× PCR buffer, 1.5 mM Magnesium chloride, 200 μM dNTP mix, 1 μM each primer, 10% DMSO, 2.5 units Amplitaq Gold Taq DNA polymerase (Perkin Elmer)and 5 μl human marathon ready cDNA (Clontech) in a total volume of 50 μl. The human cDNAs investigated for expression of ORG10 were: heart, kidney, skeletal muscle, spleen, ovary, lung, liver, thymus, testis, small intestine and brain (Clontech). A positive control reaction with human genomic DNA (Promega) was also set up. PCR amplification of the housekeeping gene G3PDH was performed as described above using sequence-specific primers purchased from Clontech, and this was used as a positive control for each cDNA template. Reactions were cycled in a MJ Research PTC-200 Thermal Cycler using the following conditions: 94° C., 5 min and 33 cycles of 94° C. for 1 min, 55° C. for 1 min 30 sec, 72° C. for 2 min, followed by an extension of 72° C. for 10 min. PCR products were separated on 1% agarose gels containing ethidium bromide (10 mg/ml) and visualised under UV light.

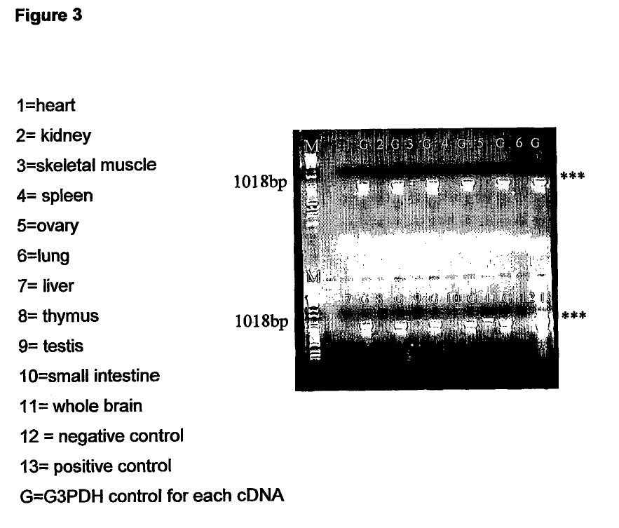

Following 33 cycles of amplification an intense band of 1104 bp corresponding to the full length ORG10 cDNA was observed in brain, with very faint bands present in heart, skeletal muscle, lung, liver and testis. (FIG. 3).

CNS Tissue Distribution of ORG10 by in situ Hybridisation

The CNS distribution profile of ORG10 was investigated in coronal and transverse sections of rat brain utilising ³³P-labelled oligonucleotide probes targeted against ORG10. Male Sprague-Dawley rats (Harlan Olac, UK) weighing 200-250 g were sacrificed by cervical dislocation. Brains were removed and frozen in isopentane cooled to −35° C. using dry ice and stored at 80° C. until further use. 20 m coronal sections were cryostat-cut onto superfrost slides (BDH) throughout the rostrocaudal extent of the rat brains. For coronal sections, a series of slides (with 3 sections on each slide) were collected from 4.7 mm through to −14.08 mm from Bregma. For transverse sections, a series of slides (with 2 sections on each slide) were collected from 3.1 mm to 7.6 mm from Bregma (according to the atlas of Paxinos and Watson, 1982). Sections were stored at −80° C. until use for a maximum of six weeks.

A synthetic oligonucleotide probe was designed from the human ORG10 sequence to also be complementary to the rodent orthologue of ORG10. The specificity of this oligonucleotide probe was assessed using the National Centre for Biotechnology Information (NCBI) database utilising a Basic Local Alignment Search Tool (BLAST, Altschul et al., 1990) in order to verify that the selected sequence was non-complementary to any other known sequences.

The 45-mer oligonucleotide selected (sequence 5′-TGG CCG GAG GGG CGG CAA GAA GGA GAG GCG GCT GTC CAG AGA GTC-3′) was 90% homologous to the human ORG10 sequence and demonstrated homology to bases 695-652 of SEQ ID NO: 4.

3′-end labelling of the oligonucleotide probe was carried out using ³³P-dATP. The oligonucleotide probe, at a concentration of 4 pmol, was radio-labelled by the isotope at an incubation temperature of 37° C. in a mixture containing, 8 μl terminal transferase reaction buffer, 2 μl terminal deoxynucleotide transferase pH 7.2 (all reagents Promega) with 3.2 μl of ³³P-dATP (3000 Ci/mmol, NEN) and made up to a final volume of 40 μl with diethylpyrocarbonate (DEPC)-treated H₂O. Following 1 hour incubation, the reaction was stopped by heating the reaction mixture to 70° C. The labelled probe was subsequently purified using Micro Bio-Spin chromatography columns (P-30 Tris RNase-free, Bio-Rad) centrifuged at 4000 r.p.m. for 4 minutes (microcentrifuge 5415C, Eppendorf). 1 μl of the probe was removed and counted by a liquid scintillation counter (Tricarb, 1500 Packard) beta-counter to assess the efficiency of radioactive labelling. Only oligonucleotide probes with counts in the range 100,000-300,000 cpm/μl were utilised for further hybridisation studies.

Sections were removed from the −80° C. freezer and thawed at room temperature for 30 minutes. Sections were then fixed in a 4% paraformaldehyde solution for 10 minutes, rinsed in DEPC treated water and incubated in 0.25% solution of acetic anhydride in 0.1 M triethalolamine and 0.9% saline (pH 8.0) for a further 10 minutes. Sections were then dehydrated in a series of ascending concentrations of ethanol (1 min in 70%, 1 min in 80%, 2 min in 90%, and 1 min in 100%), defated for 5 minutes in 100% chloroform, rehydrated for 1 min in 100% and 1 min in 95% ethanol and finally air dried at room temperature. Slides were then placed in an air-tight hybridisation box containing paper towels soaked in 50% formamide. Labelled probes were added to hybridisation solution (50% formamide, 4× standard sodium citrate (SCC), 10% dextran sulphate and 10 mM dithiothreitol, 0.2 mg/ml denatured salmon testes DNA, 0.1% Polyadenylic acid, all reagents Sigma), to a final concentration of 0.5 pmol of hybridisation solution. 150 μl of this solution was added to each of the slides, which were then covered with Hybrislips (Sigma) and incubated in an incubation oven (Mini 10, Hybaid) at 42° C. for 18 hours. Following the incubation period, Hybrislips were floated off using 2×SSC and the slides were returned to racks for further processing. Stringent washes were carried out for 30 minutes at room temperature in 1×SSC, 30 minutes at 55° C. in 1×SSC, and 10 minutes at 55° C. in 0.1×SSC. Sections were then dehydrated for 2 minutes in 70% and 95% ethanol and allowed to air dry. Once dry, the sections were placed in a cassette and exposed to X-ray film (BioMax MR-1, Kodak) for 21 days at RT. An autoradiographic ¹⁴C micro-scale (Amersham) of known radioactivity (range 31-833 nCi/g) was also placed in each cassette. The film was then developed in using a Kodak automatic developer (M-35M X-OMAT Processor).

Specificity of the oligonucleotide probes was further assessed by RNAse pretreatment of the sections or by the addition of 100-fold excess non-labelled probe. After fixation in paraformaldehyde and incubation in acetic anhydride, brain sections were incubated in RNAse A solution (ribonuclease A (Sigma) 10 μg/ml) at 37° C. for 30 minutes prior to hybridisation. The sections were then processed for in situ hybridisation as previously described. For controls involving excess non-labelled probe, 50 pmol of non-labelled ORG10 oligonucleotide probe was added to the appropriate hybridisation buffer prior to hybridisation. In situ hybridisation was then performed as previously described. No specific in situ hybridisation signal was observed following pre-treatment of the sections with RNAse A or following competition by addition of 100-fold excess non-labelled oligonucleotide probe.

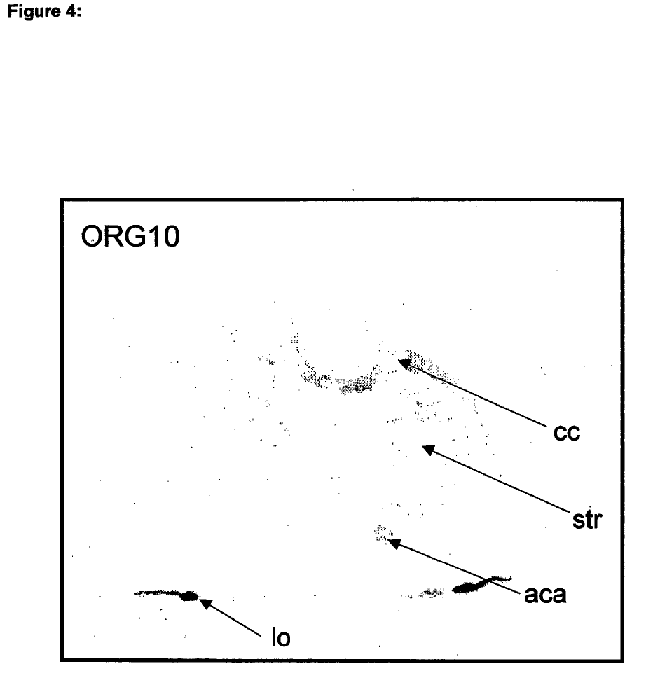

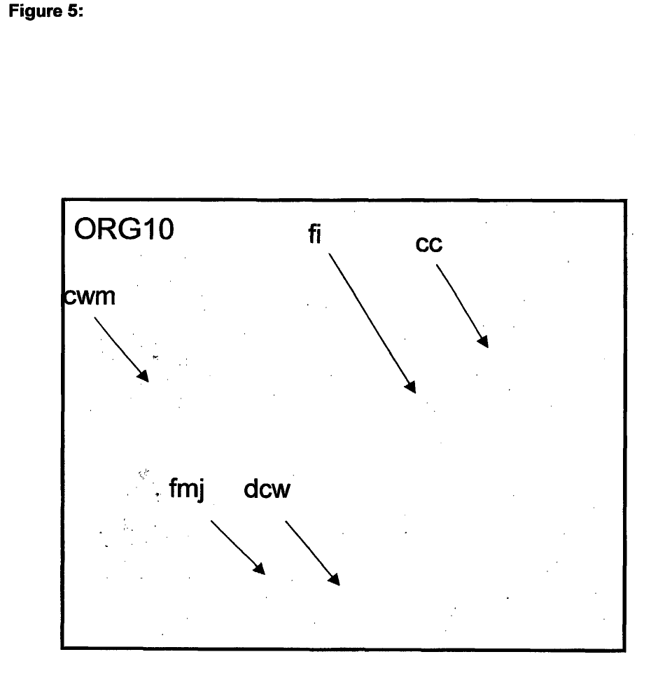

Densitometric analysis of autoradiographs was performed using a Microcomputer Imaging Device (MCID) system. Optical density measurements were obtained from coronal levels throughout the rostrocaudal extent of each rat brain. High levels of expression of ORG10 signal were restricted predominantly to white matter structures such as the forceps minor and major corpus callosum, anterior commussure, intrabulbar, lateral olfactory tubercle, genu corpus callosum, anterior commisure, corpus callosum, white matter tracts throughout the striatum, fimbria hippocampus, internal capsule, formix, stria medullaris thalamus, deep cerebral white matter, mammillothalamic tract, optic tract, superior thalamic radiation and white matter tracts of the cerebellar cortex (FIGS. 4, 5).

Legends to the Figures

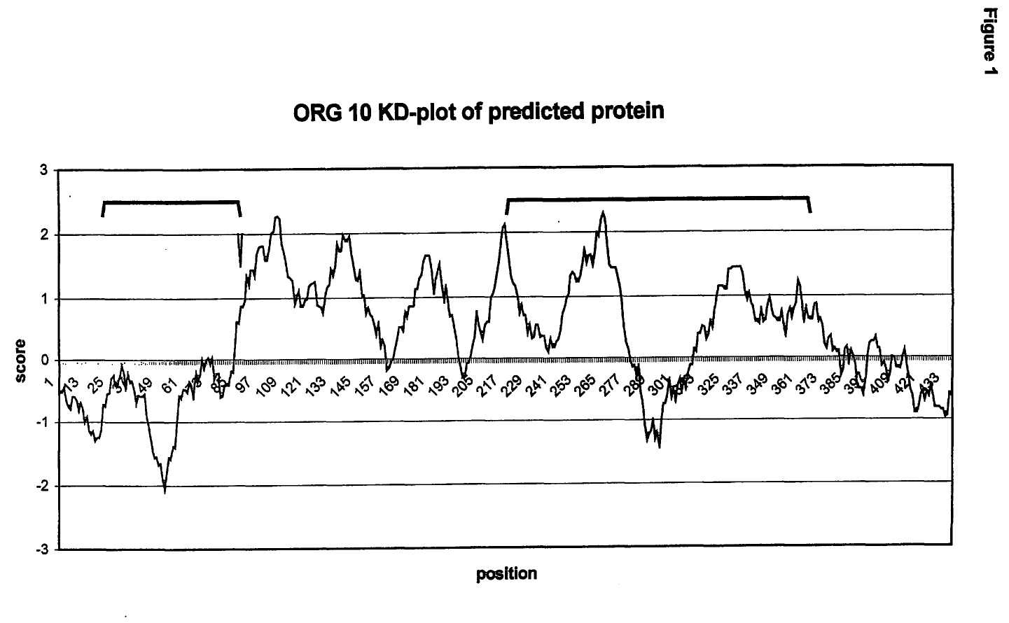

FIG. 1: Kyte-Doolittle hydrophobicity plot of predicted protein of ORG 10, showing the 7 transmembrane domains, the first 5 of which are separated from the last 2 by the long intracellular loop. The red lines indicate the areas covered by Incyte templates 077146.1 (covering 5′UTR and first few amino acids) and 446715.1 (covering TMs 4 to 7). ORF upstream of predicted methionine start site is notionally translated. The bright green arrow indicates the predicted actual translational start site. The plot end precisely at the predicted stop codon, ie. there is no notional translation in the untranslated 3′-terminus.

Detailed CNS ISH demonstrates expression of ORG10 in corpus callosum and areas of white matter indicating a potential role for ORG10 in neuronal support cells such as glia. Increasing evidence exists to support a role for glia in addition to a role in neuronal support. Glial cells have been shown to actively modulate neuronal synaptic transmission (Smit et al., 2001) and enhance CNS synaptic formation and function (Tembumi et al., 2001), suggesting a role for glia in the regulation of essential brain functions. Expression of ORG10 in macroglia may suggest that this receptor may have a role in the additional functions of these cells.

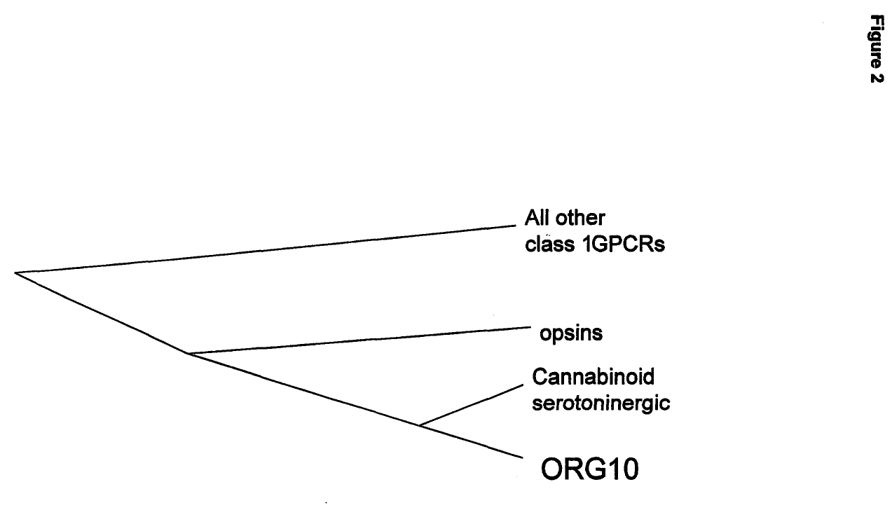

FIG. 2: Diagrammatic representation of phylogenetic relationship between ORG10 and its relatives

ORG10 is not closely related to any other known orphan GPCRs.

The branch lengths in this diagram are not to scale, and are not indicative of any quantitative measure of evolutionary distance

FIG. 3: The tissue cDNAs investigated using the ORG10 specific primers which yield a 1104 bp PCR product are labelled 1-11. The corresponding 900 bp G3PDH postive control reaction for each cDNA sample is denoted by G.

FIG. 4. Autoradiographic images of in situ hybridisation demonstrating the distribution of ORG10 mRNA in a coronal section of rat brain at 0.2 mm from Bregma according to the atlas of Paxinos and Watson (1982). ORG10 signal is localised predominantly to white matter structures such as the corpus callosum (cc), anterior commissure (aca), lateral olfactory tubercle (lo) and white matter fibre tracts throughout the striatum (str).

FIG. 5. Autoradiographic images of in situ hybridisation demonstrating the distribution of ORG10 mRNA in a transverse section of rat brain at 3.6 mm from Bregma according to the atlas of Paxinos and Watson (1982). ORG10 signal is localised predominantly to white matter structures such as the corpus callosum (cc), fimbra hippocampus (fi), deep cerebral white matter (dcw), forceps major corpus callosum (fmj) and matter fibre tracts throughout the cerebellum (cwm).

### REFERENCES

1. Altschul S F, Gish W, Miller W, Myers E W, Lipman D J (1990) J Mol Biol 215(3):403-10

2. Civelli O, Reinscheid R K, Nothacker, H P. (1999) Brain Research 848:(2):63-65.

3. Dayhoff, M. D., Atlas of protein sequence and structure, Nat. Biomed. Res. Found., Washington D.C. (1978) vol. 5, suppl. 3

4. Henikoff S, Henikoff J G. (1992) Proc Natl Acad Sci USA. 89 (22):10915-9.

5. Hinuma, S., Habata, Y., Fujii, R., Kawamata, Y., Hosoya, M., Fukusumi, S., Kitada, C., Masuo, Y., Asano, T., Matsumoto, H., Sekiguchi, A., Kurokawa, T., Nishimura, O., Onda, M., Fujino, M. (1998). Nature 393:272-276.

6. Lefkowitz, (1991) Nature, 351:353-354.

7. Lipman and Pearson (1985) Science, 227, 1435-1441

8. Meltzer H Y (1999) Neuropsychopharmacology. 21(2 Suppl):106S-115S.

9. Murphy, A. J., Paul, J. I. & Webb, D. R. Drug Discovery & Development 1, 192199 (1998).

10. Needleman S B and Wunsch C D (1970) J Mol Biol 48(3):443-53.

11. Paxinos G, Watson C. The rat brain in stereotaxic coordinates. Academic Press Limited, 1982

12. Pearson W R. (1990) Methods Enzymol 183:63-98

13. Reinscheid, R K., Nothaker, H. P., Bourson, A., Ardati, A., Henningensen, R. A., Bunzow, J. R., Grandy, D. K., Langen, H., Monsama, F. J., Civelli, O. (1995). Science 270:792-794.

14. Saiki et al., (1986) Nature 324: 163-166

15. Sakurai, T., Amemiya, A, lshii, M., Matsuzaki, I., Chemeli, R. M., Tanaka, H., Williams, S. C., Richardson, J. A., Kozlowski, G. P., Wison, S., Arch, J. R. S., Buckingham, R. E., Haynes, A. C., Carr, S. A., Annan, R. S., McNulty, D. E., Liu, W. S., Terret, J. A., Elshourbagy, N. A., Bergsma, D. J., Yanagisawa, M (1998). Cell 92:573-585.

16. Sambrook et al. (1989), Molecular Cloning: A laboratory Manual, 2^{nd} Ed., Cold Spring Harbor Laboratory, Cold Spring Harbor, N.Y.

17. Shinkai T. Ohmori O. Kojima H. Terao T. Suzuki T. Abe K (1999). American Journal of Medical Genetics. 88(2):120-2.

18. Simon et al.,(1991) Science, 252:802-808.

19. Smit, A. B., Syed, N. I., Schaap, D., Minnen, J., Klimperman, J., Kits, K. S., Lodder, H., Schors, R. C., Elk, R., Sorgedrager, B., Brejc, K., Sixma, T. K & Geraerts, W. P. M. (2001). A glia-derived acetylcholine-binding protein that modulates synaptic transmission. Nature 411:261-268.

20. Smith T F, Waterman M S (1981) J Mol Biol 147(1):195-7

21. Tembumi, M. K., & Jacob, M. H. (2001). New functions for glia in the brain. PNAS 98:3631-3632.

22. Wilson S, Bergsma D J, Chambers J K, Muir A I, Fantom K G, Ellis C, Murdock P R, Herrity N C, Stadel J M. (1998) Br J Pharmacol 125(7):1387-92.

23. Yu, Y W, Tsai, S J, Lin, C H, Hsu, C P, Yang, K H, Hong, C J (1999) Neuroreport 10(6): 1231-3.

1 A substantially pure polynucleotide, encoding the amino acid sequence of SEQ ID NO: 3 or its isoforms.

2 Polynucleotide according to claim 1, comprising the sequence according to SEQ ID NO: 4.

3 A recombinant expression vector comprising the polynucleotide according to claim 1 or 2 or fragments thereof.

4 A polypeptide according to SEQ ID NO: 3 or its isoforms.

5 Cell line transformed with a polynucleotide encoding at least part of the polypeptide according to claim 4

6 Cell line transformed with a polynucleotide according to claim 1 or 2 or fragments thereof or transformed with the expression vector of claim 3.

7 Cell line according to claim 6 of mammalian origin

8 Cell line according to claim 6 or 7 expressing an ORG10 gene product, wherein ORG10 gene is defined as a stretch of DNA hybridisable to the polynucleotide sequence according to SEQ ID NO: 1 and/or SEQ ID NO: 4

9 Use of a polynucleotide hybridisable to the ORG10 gene in the in vitro diagnosis of a psychiatric disorder, wherein ORG10 gene is defined as a stretch of DNA hybridisable to the polynucleotide sequence according to SEQ ID NO: 1 and/or SEQ ID NO: 4

10 Use of a cell line according to claim 6 to 8 in the in vitro diagnosis of a psychiatric disorder.

11 Use of a polypeptide encoded by a polynucleotide comprising SEQ ID NO: 4 or fragments thereof in the in vitro diagnosis of a psychiatric disorder.

12 Use of a polynucleotide according to claims 1 or 2 or fragments thereof or the expression vector of claim 3 in a screening assay for the identification of new drugs.

13 Use of a polypeptide according to claim 4 or analogues or fragments thereof in a screening assay for the identification of drugs for the treatment of psychiatric disorders.

14 Use of a cell line according to claims 6 to 8 in a screening assay for the identification of new drugs for the treatment of psychiatric disorders.

15 A polynucleotide comprising SEQ ID NO: 1 or fragments thereof for use as a medicament.

16 A polypeptide encoded by a polynucleotide comprising SEQ ID NO: 4 or fragments thereof for use as a medicament

17 A polynucleotide comprising SEQ ID NO: 4 or fragments thereof for use as a medicament for the treatment of a psychiatric disorder.

18 A polypeptide encoded by a polynucleotide comprising SEQ ID NO: 4 or fragments thereof for use as a medicament for the treatment of a psychiatric disorder

19 Use of a polynucleotide comprising SEQ ID NO: 4 or fragments thereof in the preparation of a medicament for the treatment of a psychiatric disorder

20 Use of a polypeptide encoded by a polynucleotide comprising SEQ ID NO: 4 or fragments thereof in the preparation of a medicament for the treatment of a psychiatric disorder

21 Antibodies against the polypeptide according to claim 4

22 Method for the detection of a mutation in the ORG10 gene in a given subject comprising the steps of
a) providing a set of oligonucleotide primers capable of hybridising to the nucleotide sequence of the ORG10 gene
b) obtaining a sample containing nucleic acid from the subject
c) amplifying a region flanked by the primer set of step 1 using a nucleic acid amplification method
d) detecting whether the amplified region contains a mutation by
e) comparing the amplified sequence with the sequence of normal control subjects.
wherein ORG10 gene is defined as a stretch of DNA hybridisable to the polynucleotide sequence according to SEQ ID NO: 1.

23 A method for identifying ligands for ORG10 gene products, said method comprising the steps of:
a) introducing into a suitable host cell a polynucleotide according to claims 1 or 2 or an expression vector according to claim 3 or fragments thereof,
b) culturing cells under conditions to allow expression of the DNA sequence
c) optionally isolating the expression product
d) bringing the expression product (or the host cell from step b)) into contact with potential ligands which will possibly bind to the protein encoded by said DNA from step a);
e) establishing whether a ligand has bound to the expressed protein.
f) Optionally isolating and identifying the ligand

24 Compounds selected with a method according to claim 23 useful in the treatment of CNS disorders.

25 Use of compounds according to claim 24 for the manufacture of a medicament useful in the treatment of CNS disorders.
